SEQUENCE LISTING (4 sequences)

SEQ ID NO: 1 - DNA, 400 bp, synthetic construct
  source 1..400: mol_type = other DNA; note = DNA sequence of a 3' UTR, T-Zm.GST314.
tccttctggaggccgccggcggtgttttggttttgtactatatctgtttttctgtctgttttgtgtactggtgccagcaaagggagcttgctttgttgaacctagtttctgtaatttgcttcgtgtgatcaagaattgtatctgctgcacagttgtaaactgtaatatatttgaataattaataaattttattttgtcataccctggagatatctgcacgcacggttttgttgctttggtttggtttgcgacttgtctgtttgggagacaataagctagctgcttgaaattttggttatattttccccacaagaaatttgtttggtgaatctctcgtcttatataataaaaacggcttgtgtttgtttttttggttgctactgtttggagcaaacaccag

SEQ ID NO: 2 - DNA, 1213 bp, Arabidopsis thaliana
  source 1..1213: mol_type = genomic DNA; note = DNA sequence of a regulatory expression element group or EXP, EXP-At.Cyco comprised of the native promoter, operably linked 5' to the native leader, operably linked 5' to the native first intron derived from the Arabidopsis thaliana Cytochrome c oxidase subunit VIa gene.
tgcgagtgggcgaattccggagcactctgattggctgaaaaaatagaaatagtagtgatgttgctcctcctctcctcctctattattaatttttcgtcgttcttcttctgaaagttgtgtggtttttagaggtcaccaaaaaaaatctattttgagatactaaaaatatttcgttttgcattttgttgtgcagccatttgttacacaggttgaagcttataactgaaaattggattcaaagaatcgtagatgaagaaatcgaagtgagttgaatattttctgaacatatgaaaattggaacaagttttttctcattttgctagtttcctgtttttatgttttcttgactttaggagatgacatatggaggtgaactatacaaaggttgttgcaacgataacattctccttaattcagtttttgcaactcggttacaagcactcagtggacttttggccaagacaattttttttttttttttctctctctctaaaatgttatagatacgaatcctttgttgaataaaggaaaaagttgaacatttgattacacataagactttaacataatccaacttttttttatatgaagctacaaacaagatttaaaacatcaaagattccatctaaacttcattcatcttcaatcttcaacatccttcaatgactagtatgtatgtacataagtaaaattgttgataagaaaacaaaacaatgatgggctaaaatagcccataaaaggcccattaaacttgggtttagactttagattcaacgacgccagattagtgagtcacataaccctcttggaaagagtctcaacacttgcagagaaaaagaacaaggaagatcccggaaacggaccgcaggtaatttctctcctctctatttttaccattttccattgacgacgatctaggttttctgatttgattttggagaacgcctcgatgagtttatagattcgtagattggttttgagattcagtataatttcacccggattccaatttttgaaccgatacctaattttgaattgatttggtagatcgattggtcaaatttgaaattgatttttctccataatatctgaagcgtcttattggatcaaatctacaacatttctctgttgaaaggatcgattttttttttcttggaacatgataacttttgattattcatcaaagttttgttctttttaatatttcacaggt

SEQ ID NO: 3 - DNA, 2001 bp, synthetic construct
  source 1..2001: mol_type = other DNA; note = Synthetic coding sequence used for plant expression for beta-glucuronidase (GUS) with a processable intron derived from the potato light-inducible, tissue-specific St-LS1 gene (GenBank Accession: X04753).
atggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatga

SEQ ID NO: 4 - DNA, 315 bp, Gossypium barbadense
  source 1..315: mol_type = genomic DNA; note = DNA sequence of a 3' UTR, T-Gb.FbL2 derived from the Gossypium barbadense fiber FbLate-2 gene.
accatatgacactggtgcatgtgccatcatcatgcagtaatttcatggtatatcttaattatatggttaataaaaaaaagatggtgagtgaataatgtgcgtgcattcctccatgcaccaatggtgaatctctttgcatacatagagattctgaatgattatagtttatgttgtagtgaaattaattttgaatgttgtttttaaattttaatgtcacttggcttgatttatgttttaacgaagcttatgttatgtattttactttaatgatattgcatgtattgttaatttaacattgcttgatcagtatactct

(19) United States
(12) Patent Application Publication
(10) Pub. No.: US 2025/0160276 A1
(43) Pub. Date: May 22, 2025

(54) PLANT REGULATORY ELEMENTS AND USES THEREOF

(71) Applicant: Monsanto Technology LLC, St. Louis, MO (US)

(72) Inventor: Krishnakumar Sridharan, Cary, NC (US)

(21) Appl. No.: 18/949,035

(22) Filed: Nov. 15, 2024

Related U.S. Application Data
(60) Provisional application No. 63/601,354, filed on Nov. 21, 2023.

(51) Int. Cl. A01H 1/00; C12N 5/04; C12N 15/113

(52) U.S. Cl. CPC A01H 1/123; A01H 1/1245; C12N 5/04; C12N 15/113; C12N 2510/00

(57) ABSTRACT

Recombinant DNA polynucleotides and constructs, as well as their nucleotide sequences, useful for modulating gene expression in plants are provided. Also, transgenic plants, plant cells, plant parts, and seeds comprising the recombinant DNA polynucleotides operably linked to heterologous transcribable DNA polynucleotides are provided. Also provided are methods of the use of the recombinant DNA polynucleotides and constructs.

### REFERENCE TO RELATED APPLICATIONS

This application claims the benefit of U.S. Provisional Application No. 63/601,354, filed Nov. 21, 2023, herein incorporated by reference in its entirety.

### INCORPORATION OF SEQUENCE LISTING

The sequence listing that is contained in the file named “MONS584US_ST26.xml”, is 9,601 bytes (as measured in Microsoft Windows®), was created on Oct. 31, 2024, and is filed contemporaneously by electronic submission (using the United States Patent Office Patent Center) and incorporated by reference in its entirety.

### FIELD

The present disclosure relates to the field of plant molecular biology and plant genetic engineering. More specifically, the present disclosure relates to DNA polynucleotides useful for modulating gene expression in plants.

### BACKGROUND

Regulatory elements are genetic elements that regulate gene activity by modulating the transcription of an operably linked transcribable DNA polynucleotide. Such elements may include promoters, leaders, introns, and 3′ untranslated regions (3′ UTRs) and are useful in the field of plant molecular biology and plant genetic engineering.

### SUMMARY

This section provides a general summary of the disclosure and is not a comprehensive disclosure of its full scope or all of its features.

Provided herein are gene regulatory elements for use in plants. Several embodiments relate to recombinant DNA polynucleotides comprising the regulatory elements. Also provided are transgenic plant cells, plants, parts thereof and seeds comprising the regulatory elements. In one embodiment, the regulatory elements are operably linked to a transcribable DNA polynucleotide. In certain embodiments, the transcribable DNA polynucleotide may be heterologous with respect to the regulatory DNA sequence. Thus, a regulatory element DNA sequence provided herein may, in particular embodiments, be defined as operably linked to a heterologous transcribable DNA polynucleotide. Several embodiments relate to methods of using the regulatory elements and making and using the recombinant DNA polynucleotides comprising the regulatory elements, and the transgenic plant cells, plants, and seeds comprising the regulatory elements operably linked to a transcribable DNA polynucleotide.

In some embodiments, a recombinant DNA polynucleotide comprising a DNA sequence selected from the group consisting of: (a) a sequence with at least about 85 percent sequence identity to SEQ ID NO:1; (b) a sequence comprising SEQ ID NO:1; and (c) a fragment of SEQ ID NO:1, wherein the fragment comprises gene regulatory activity; wherein the DNA sequence is operably linked to a heterologous transcribable DNA polynucleotide is provided. In specific embodiments, the recombinant DNA polynucleotide comprises a DNA sequence having at least about 85 percent, at least about 86 percent, at least about 87 percent, at least about 88 percent, at least about 89 percent, at least about 90 percent, at least 91 percent, at least 92 percent, at least 93 percent, at least 94 percent, at least 95 percent, at least 96 percent, at least 97 percent, at least 98 percent, or at least 99 percent sequence identity to the DNA sequence of SEQ ID NO:1.

In another aspect, provided herein are transgenic plant cells comprising a recombinant DNA polynucleotide comprising a DNA sequence selected from the group consisting of: (a) a sequence with at least about 85 percent sequence identity to SEQ ID NO:1; (b) a sequence comprising SEQ ID NO:1; and (c) a fragment of SEQ ID NO:1, wherein the fragment comprises gene regulatory activity; wherein the DNA sequence is operably linked to a heterologous transcribable DNA polynucleotide. In certain embodiments, the transgenic plant cell may be a monocotyledonous plant cell. In other embodiments, the transgenic plant cell may be a dicotyledonous plant cell.

In still yet another aspect, further provided herein is a transgenic plant, or part thereof, comprising a recombinant DNA polynucleotide comprising a DNA sequence selected from the group consisting of: a) a sequence with at least 85 percent sequence identity to SEQ ID NO:1; b) a sequence comprising SEQ ID NO:1; and c) a fragment of SEQ ID NO:1, wherein the fragment comprises gene regulatory activity; wherein the DNA sequence is operably linked to a heterologous transcribable DNA polynucleotide. In specific embodiments, the transgenic plant may be a progeny plant of any generation that comprises the recombinant DNA polynucleotide. A transgenic seed comprising the recombinant DNA polynucleotide that produces such a transgenic plant when grown is also provided.

Several embodiments relate to a method of producing a commodity product comprising obtaining a transgenic plant or part thereof containing a recombinant DNA polynucleotide as described herein, such as those comprising a DNA sequence selected from SEQ ID NO:1 or fragments or variants thereof and producing the commodity product therefrom. In one embodiment, the commodity product may be seeds, processed seeds, protein concentrate, protein isolate, starch, grains, plant parts, seed oil, biomass, flour and/or meal.

Several embodiments relate to a method of producing a transgenic plant comprising a recombinant DNA polynucleotide as described herein, comprising transforming a plant cell with the recombinant DNA polynucleotide to produce a transformed plant cell and regenerating a transgenic plant from the transformed plant cell. In a further embodiment, a method of expressing a transcribable DNA polynucleotide is provided, the method comprising obtaining a transgenic plant as described herein and cultivating said plant, wherein the transcribable DNA polynucleotide is expressed.

### BRIEF DESCRIPTION OF THE SEQUENCES

SEQ ID NO:1 is a 400-nucleotide DNA sequence of a synthetic 3′ UTR, T-Zm.GST314.

SEQ ID NO:2 is a 1,213-nucleotide DNA sequence of a regulatory expression element group or EXP, EXP-At.Cyco, comprised of the native promoter, operably linked 5′ to the native leader, operably linked 5′ to the native first intron derived from the Arabidopsis thaliana Cytochrome c oxidase subunit VIa gene (locus AT4G37830.3).

SEQ ID NO:3 is a 2,001-nucleotide synthetic coding sequence used for plant expression for β-glucuronidase (GUS) with a processable intron derived from the potato light-inducible, tissue-specific St-LS1 gene (GenBank Accession: X04753).

SEQ ID NO:4 is a 315-nucleotide DNA sequence of a 3′ UTR, T-Gb.FbL2 derived from the Gossypium barbadense fiber FbLate-2 gene (GenBank Accession U34401.1).

### DETAILED DESCRIPTION

Example embodiments will now be described more fully. The description and specific examples included herein are intended for purposes of illustration only and are not intended to limit the scope of the present disclosure. The following definitions, descriptions, and methods are provided to better define the invention and to guide those of ordinary skill in the art in the practice of the invention. Unless otherwise noted, terms are to be understood according to conventional usage by those of ordinary skill in the relevant art.

Provided herein are regulatory elements having gene regulatory activity in plants. A nucleotide sequence of such a regulatory element is provided as SEQ ID NO:1. These regulatory elements are capable of affecting the expression of an operably linked transcribable DNA polynucleotide in plant tissues, and therefore regulating gene expression of an operably linked transgene in transgenic plants. Also provided are methods of modifying, producing, and using recombinant DNA polynucleotides which contain the provided regulatory elements. Also provided are compositions that include transgenic plant cells, plants, plant parts, and seeds containing the recombinant DNA polynucleotides comprising one or more regulatory elements as described herein, and methods for preparing and using the same.

### DNA Polynucleotides

DNA polynucleotides, fragments and variants thereof, and their corresponding DNA sequences are provided. As used herein, the terms “DNA”, “DNA polynucleotide”, “DNA molecule” and “nucleic acid molecule” refer to a deoxyribonucleic acid (DNA) molecule. A DNA polynucleotide may be described by convention from the 5′ (upstream) end to the 3′ (downstream) end. A DNA polynucleotide may be of genomic or synthetic origin and/or comprise a recombinant or heterologous DNA polynucleotide or sequence. As used herein, the term “DNA sequence” refers to the nucleotide sequence of a DNA polynucleotide, i.e. the sequence of consecutive nucleotides in the DNA molecule. Examples of DNA sequences disclosed herein include the DNA sequences of SEQ ID NOs:1-4, or variants or fragments thereof, or the reverse complements of any of SEQ ID NOs:1-4. As used herein in reference to nucleotides of a DNA polynucleotide or DNA sequence or molecule, the terms “consecutive” and “contiguous” are interchangeable and synonymous and refer to linked nucleotides in a DNA polynucleotide or DNA sequence, strand or molecule without any gap or interruption between them. The nomenclature used herein corresponds to that of Title 37 of the United States Code of Federal Regulations § 1.822 and set forth in WIPO Standard ST.26 (2021), Annex I, Tables 1 and 3.

As used herein, a “recombinant DNA polynucleotide” is a DNA polynucleotide comprising a combination of DNA polynucleotides that would not naturally occur together without human intervention. For instance, a recombinant DNA polynucleotide may be a DNA polynucleotide that is comprised of at least two DNA polynucleotides heterologous with respect to each other, or a DNA polynucleotide that comprises a DNA sequence that deviates from DNA sequences that exist in nature, or a DNA polynucleotide that comprises a synthetic DNA sequence or a DNA polynucleotide that has been incorporated into a host cell's DNA by genetic transformation or gene editing.

As used herein, a “synthetic nucleotide sequence” or “artificial nucleotide sequence” is a nucleotide sequence that is not known to occur in nature or that is not naturally occurring. Preferably, synthetic nucleotide sequences share little or no extended homology to natural nucleotide sequences. Extended homology in this context generally refers to 100% sequence identity extending beyond about 25 nucleotides of contiguous sequence. An example of a synthetic nucleotide sequence is the 3′ UTR, T-Zm.GST314 (SEQ ID NO:1).

Reference in this application to an “isolated DNA polynucleotide”, or an equivalent term or phrase, is intended to mean that the DNA polynucleotide is one that is present alone or in combination with other compositions, but not within its natural environment. For example, nucleic acid elements such as a coding sequence, intron sequence, untranslated leader sequence, promoter sequence, transcriptional termination sequence (i.e., 3′ UTR), and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding an insecticidal protein or any naturally occurring insecticidal variant of that protein would be an isolated nucleotide sequence so long as the nucleotide sequence was not within the DNA of the bacterium from which the sequence encoding the protein is naturally found. A synthetic nucleotide sequence encoding the amino acid sequence of the naturally occurring insecticidal protein would be considered to be isolated for the purposes of this disclosure. For the purposes of this disclosure, any transgenic nucleotide sequence, e.g., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As used herein, the term “sequence identity” refers to the extent to which two optimally aligned polynucleotide sequences or two optimally aligned polypeptide sequences are identical. An optimal sequence alignment is created by aligning two sequences, e.g., a reference sequence and another sequence, to maximize the number of nucleotide matches in the sequence alignment with appropriate internal nucleotide insertions, deletions, or gaps. In some embodiments, a DNA sequence provided as SEQ ID NO:1 is used as the reference sequence.

As used herein, the terms “percent sequence identity”, “% sequence identity”, “percent identity” and “% identity” are the identity fraction multiplied by 100. The “identity fraction” for a sequence optimally aligned with a reference sequence is the number of nucleotide matches in the optimal alignment, divided by the total number of nucleotides in the reference sequence, e.g., the total number of nucleotides in the full length of the entire reference sequence. Thus, several embodiments relate to a DNA polynucleotide comprising a DNA sequence that, when optimally aligned to a reference sequence, provided herein as SEQ ID NO:1, has at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, at least about 99 percent identity, or at least about 100 percent identity to the reference sequence. A DNA sequence as disclosed herein may have the activity of the reference sequence from which it is derived, for example it may have the activity of SEQ ID NO:1. Also, a DNA sequence as provided herein may comprise the same or similar activity as the reference sequence from which it is derived, for example it may comprise the same or similar activity as SEQ ID NO:1.

### Regulatory Elements

Regulatory elements such as promoters, leaders (also known as 5′ UTRs), enhancers, introns, and transcription termination regions (also known as 3′ UTRs) play an integral part in the overall expression of genes in living cells. The terms “regulatory element”, “gene regulatory element”, “expression element” and “regulatory expression element” as used herein, refer to a DNA polynucleotide having gene regulatory activity. The term “transcriptional regulatory element” as used herein, refers to a DNA polynucleotide having gene regulatory activity by affecting the transcription of a gene operably linked to it. The term “gene regulatory activity,” as used herein, refers to the ability to affect the expression of an operably linked transcribable DNA polynucleotide, for instance by affecting the transcription and/or translation of the operably linked transcribable DNA polynucleotide. Regulatory elements, such as promoters, leaders, enhancers, introns and 3′ UTRs that function in plants are useful for modifying plant phenotypes through genetic engineering.

As used herein, a “regulatory expression element group” or “EXP” sequence may refer to a group of operably linked regulatory elements, such as enhancers, promoters, leaders, and introns. For example, a regulatory expression element group may be comprised, for instance, of a promoter operably linked 5′ to a leader sequence, operably linked 5′ to an intron sequence.

As used herein in reference to regulatory elements, the terms “fragment” and “functional fragment” are interchangeable and refer to a fragment or portion of a regulatory element, that affects, modulates or drives the expression of an operably linked transcribable DNA polynucleotide i.e., that has gene regulatory activity. A fragment of a regulatory element may affect, modulate or drive expression of an operably linked transcribable DNA polynucleotide in a similar manner as the reference sequence from which it is derived. According to one embodiment of the present disclosure, a fragment of SEQ ID NO:1 is provided that may have gene regulatory activity. In another embodiment, a fragment of SEQ ID NO:1 is provided that may comprise the same or similar gene regulatory activity as the reference sequence from which it is derived i.e., SEQ ID NO:1. In alternative embodiments, gene regulatory fragments of SEQ ID NO:1 are provided comprising at least about 50, at least about 75, at least about 95, at least about 100, at least about 125, at least about 150, at least about 175, at least about 200, at least about 225, at least about 250, at least about 275, at least about 300, at least about 325, at least about 350, at least about 375 contiguous nucleotides of SEQ ID NO:1.

Regulatory elements may be characterized by their gene expression pattern, e.g., positive and/or negative effects such as constitutive expression or temporal, spatial, developmental, tissue, environmental, physiological, pathological, cell cycle, and/or chemically responsive expression, and any combination thereof, as well as by quantitative or qualitative indications. As used herein, a “gene expression pattern” is any pattern of transcription of an operably linked DNA polynucleotide into a transcribed RNA molecule resulting in relative levels and abundance of the transcribed RNA molecule in various plant tissues and cells during development. The transcribed RNA may be translated to produce a protein or may provide an antisense or other regulatory RNA, such as a double-stranded RNA (dsRNA), a transfer RNA (RNA), a ribosomal RNA (rRNA), a microRNA (miRNA), a small interfering RNA (siRNA), and the like.

As used herein, the term “protein expression” is any pattern of translation of a transcribed RNA into a protein. Protein expression may be characterized by its temporal, spatial, developmental, or morphological qualities, as well as by quantitative or qualitative indications.

A promoter is useful as a regulatory element for modulating the expression of an operably linked transcribable DNA polynucleotide. As used herein, the term “promoter” refers generally to a DNA polynucleotide that is involved in recognition and binding of RNA polymerase, e.g., RNA polymerase II, and other proteins, such as trans-acting transcription factors, to initiate transcription. A promoter may be initially isolated from the 5′ untranslated region (5′ UTR) of a genomic copy of a gene. In some embodiments, a promoter is operably linked 5′ to a leader sequence. Promoters may be synthetically produced or manipulated DNA polynucleotides. Promoters may also be chimeric. Chimeric promoters are produced through the fusion of two or more heterologous DNA polynucleotides.

A promoter or promoter fragment as described herein may be analyzed for the presence of known promoter elements, e.g., DNA sequence characteristics, such as a TATA box and other known transcription factor binding site motifs. Identification of such known promoter elements may be used by one of skill in the art to design variants of the promoter having a similar expression pattern to the original promoter.

As used herein, the term “leader” refers to a DNA polynucleotide isolated from the untranslated 5′ region (5′ UTR) of a gene and is defined generally as a nucleotide segment between the transcription start site (TSS) and the protein coding sequence start site. Alternately, leaders may be synthetically produced or manipulated DNA clements. A leader can be used as a 5′ regulatory element for modulating expression of an operably linked transcribable DNA polynucleotide. Leader polynucleotides may be used with a heterologous promoter or with their native promoter. In specific embodiments, such DNA sequences may be defined as being capable of acting as a leader in a host cell, including, for example, a transgenic plant cell. In one embodiment, such sequences are decoded as comprising leader activity.

As used herein, the term “intron” refers to a DNA polynucleotide that may be isolated or identified from a gene and may be defined generally as a region spliced out during messenger RNA (mRNA) processing prior to translation. Alternately, an intron may be a synthetically produced or modified DNA element. An intron may contain enhancer elements that effect the transcription of operably linked genes. An intron may be used as a regulatory element for modulating expression of an operably linked transcribable DNA polynucleotide. A construct may comprise an intron, and the intron may or may not be heterologous with respect to the transcribable DNA polynucleotide. Examples of introns include the rice actin intron and the corn HSP70 intron.

In plants, the inclusion of some introns in gene constructs leads to increased mRNA and protein accumulation relative to constructs lacking the intron. This effect has been termed “intron mediated enhancement” (IME) of gene expression. Introns known to stimulate expression in plants have been identified in maize genes (e.g., tubA1, Adh1, Sh1, and Ubi1), in rice genes (e.g., tpi) and in dicotyledonous plant genes like those from petunia (e.g., rbcS), potato (e.g., st-1s1) and from Arabidopsis thaliana (e.g., ubq3 and pat1). It has been shown that deletions or mutations within the splice sites of an intron reduce gene expression, indicating that splicing might be needed for IME. However, IME in dicotyledonous plants has been shown by point mutations within the splice sites of the pat1 gene from A. thaliana. Multiple uses of the same intron in one plant has been shown to exhibit disadvantages. In those cases, it is necessary to have a collection of basic control clements for the construction of appropriate recombinant DNA clements.

As used herein, the terms “3′ transcription termination polynucleotide,” “3′ untranslated region” and “3′ UTR” refer to a DNA polynucleotide that is used during transcription to the untranslated region of the 3′ portion of an mRNA. The 3′ untranslated region of an mRNA may be generated by specific cleavage and 3′ polyadenylation, also known as a poly(A) tail. A 3′ UTR may be operably linked to and located downstream of a transcribable DNA polynucleotide and may include a polyadenylation signal and other regulatory signals capable of affecting transcription, mRNA processing, or gene expression. Poly(A) tails are thought to function in mRNA stability and in initiation of translation. Examples of 3′ transcription termination polynucleotides are the nopaline synthase 3′ region, wheat hsp17 3′ region, pea rubisco small subunit 3′ region, cotton E6 3′ region, and the coixin 3′ UTR.

3′ UTRs typically find beneficial use for the recombinant expression of specific DNA polynucleotides. A weak 3′ UTR has the potential to generate read-through during transcription, which may affect the expression of the DNA polynucleotide located in the neighboring expression cassettes. Appropriate control of transcription termination can prevent read-through into other DNA sequences (e.g., other expression cassettes) localized downstream and can further allow efficient recycling of RNA polymerase to improve gene expression. Efficient termination of transcription (release of RNA Polymerase II from the DNA) is prerequisite for re-initiation of transcription and thereby directly affects the overall transcript level. Subsequent to transcription termination, the mature mRNA is released from the site of synthesis and transported to the cytoplasm. Eukaryotic mRNAs are accumulated as poly(A) forms in vivo, making it difficult to detect transcriptional termination sites by conventional methods. However, prediction of functional and efficient 3′ UTRs by bioinformatics methods is difficult in that there are no conserved DNA sequences that would allow easy prediction of an effective 3′ UTR.

From a practical standpoint, it is typically beneficial that a 3′ UTR used in an expression cassette possesses the following characteristics. First, the 3′ UTR should be able to efficiently and effectively terminate transcription of the transcribable DNA polynucleotide (e.g., a transgene) and prevent read-through of the transcript into any neighboring DNA sequence, which can be comprised of another expression cassette as in the case of multiple expression cassettes residing in one transfer DNA (T-DNA), or the neighboring chromosomal DNA into which the T-DNA has inserted during plant transformation. Second, the 3′ UTR should not cause a reduction in the transcriptional activity imparted by the promoter, leader, enhancers, and introns that are used to drive expression of the transcribable DNA polynucleotide. Finally, in plant biotechnology, the 3′ UTR is often used for priming of amplification reactions of reverse transcribed RNA extracted from the transformed plant and used to: (1) assess the transcriptional activity or expression of the expression cassette once integrated into the plant chromosome; (2) assess the copy number of insertions within the plant DNA; and (3) assess zygosity of the resulting seed after breeding. The 3′ UTR is also used in amplification reactions of DNA extracted from the transformed plant to characterize the intactness of the inserted cassette. A 3′ UTR useful in combination with further regulatory clements (e.g., the regulatory elements presented as SEQ ID NO:2) is provided as SEQ ID NO: 1. 3′ UTRs also useful in combination with further regulatory elements are fragments and variants of SEQ ID NO:1, wherein said fragments and variants may comprise the same or similar activity as SEQ ID NO:1.

As used herein, the term “enhancer” or “enhancer element” refers to a cis-acting regulatory element, also known as cis-element, which confers an aspect of the overall expression pattern, but is usually insufficient alone to drive transcription of an operably linked transcribable DNA polynucleotide. Unlike promoters, enhancer elements do not usually include a transcription start site (TSS) or TATA box or equivalent DNA sequence. A promoter or promoter fragment may naturally comprise one or more enhancer elements that affect the transcription of an operably linked DNA sequence. An enhancer element may also be fused to a promoter to produce a chimeric promoter cis-element, which confers an aspect of the overall modulation of gene expression.

As used herein, the term “variant” refers to a second DNA polynucleotide, such as a regulatory element, that is in composition similar, but not identical to, a first DNA polynucleotide, and wherein the second DNA polynucleotide still maintains the general functionality, e.g., the same or similar expression pattern, for instance through more or less equivalent transcriptional activity, of the first DNA polynucleotide. A variant may be a shorter or truncated version of the first DNA polynucleotide or an altered version of the sequence of the first DNA polynucleotide, such as one with different restriction enzyme sites and/or internal deletions, substitutions, or insertions. A “variant” can also encompass a regulatory element having a nucleotide sequence comprising one or more modifications for example, a substitution, duplication, deletion, and/or insertion of one or more nucleotides of a reference sequence, wherein the derivative regulatory element has more or less or equivalent transcriptional or translational activity than the corresponding parent regulatory polynucleotide or element. Regulatory element “variants” will also encompass variants arising from mutations that naturally occur in bacterial and plant cell transformation. In some embodiments, a DNA polynucleotide sequence provided as SEQ ID NO:1 may be used to create variants that are similar in composition, but not identical to, the DNA sequence of the original regulatory element i.e., of SEQ ID NO:1, while still maintaining the general functionality, e.g., the same or similar expression pattern, of the original regulatory element. Production of such variants is well within the ordinary skill of the art in light of the disclosure and is contemplated herein.

The efficacy of the modifications (such as a substitution, duplication, deletion, and/or insertion) described herein on the desired expression aspects of a particular transgene may be tested empirically in stable and transient plant assays, such as those described in the working examples herein, so as to validate the results, which may vary depending upon the changes made and the goal of the change in the starting DNA polynucleotide.

### Constructs

As used herein, the term “construct” means any recombinant DNA polynucleotide or molecule such as a plasmid, cosmid, virus, phage, or linear or circular DNA or RNA polynucleotide, derived from any source, capable of genomic integration or autonomous replication, comprising a DNA polynucleotide where at least one DNA polynucleotide has been linked to another DNA polynucleotide in a functionally operative manner, i.e., operably linked. As used herein, the term “vector” means any construct that may be used for the purpose of transformation, i.e., the introduction of heterologous DNA or RNA into a host cell. A construct typically includes one or more expression cassettes. As used herein, an “expression cassette” refers to a recombinant DNA polynucleotide comprising at least a transcribable DNA polynucleotide operably linked to one or more regulatory elements, typically at least a promoter and a 3′ UTR.

As used herein, the term “operably linked” refers to a first DNA polynucleotide joined to a second DNA polynucleotide, wherein the first and second DNA polynucleotides are so arranged that the first DNA polynucleotide affects the function of the second DNA polynucleotide. The two DNA polynucleotides may or may not be part of a single contiguous DNA polynucleotide and may or may not be adjacent. For example, a promoter is operably linked to a transcribable DNA polynucleotide if the promoter modulates transcription of the transcribable DNA polynucleotide of interest in a cell. A leader, for example, is operably linked to a DNA sequence when it is capable of affecting the transcription or translation of the DNA sequence. A 3′ UTR, for example, is operably linked to a transcribable DNA polynucleotide if the 3′ UTR modulates transcription and/or terminates the transcription of the transcribable DNA polynucleotide of interest in a cell.

In some embodiments, one or more regulatory elements as described herein operably linked to a transcribable DNA polynucleotide are provided in double tumor-inducing (Ti) plasmid border constructs that have the right border (RB or AGRtu.RB) and left border (LB or AGRtu.LB) regions of the Ti plasmid isolated from Agrobacterium tumefaciens comprising a T-DNA that, along with transfer molecules provided by the A. tumefaciens cells, permit the integration of the T-DNA into the genome of a plant cell (see, e.g., U.S. Pat. No. 6,603,061). The constructs may also contain the plasmid backbone DNA segments that provide replication function and antibiotic selection in bacterial cells, e.g., an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404, however other strains known to those skilled in the art of plant transformation can function.

Methods are known in the art for assembling and introducing constructs into a cell in such a manner that the transcribable DNA polynucleotide is transcribed into a functional mRNA that is translated and expressed as a protein. Compositions and methods for preparing and using constructs and host cells are well known to one skilled in the art. Typical vectors useful for expression of nucleic acids in plants are well known in the art and include vectors derived from the Ti plasmid of Agrobacterium tumefaciens and the pCaMVCN transfer control vector.

Various regulatory elements may be included in a construct, including any of those provided herein. Any such regulatory clements may be provided in combination with other regulatory clements. Such combinations can be designed or modified to produce desirable regulatory features. In one embodiment, constructs may comprise at least one regulatory element operably linked to a transcribable DNA polynucleotide operably linked to a 3′ UTR. A construct as disclosed herein may comprise at least one regulatory element, operably linked to a transcribable DNA polynucleotide, operably linked to SEQ ID NO:1. In another embodiment, a construct as disclosed herein may comprise at least one regulatory element, operably linked to a transcribable DNA polynucleotide, operably linked to a fragment or a variant of SEQ ID NO:1.

Expression cassettes may also include a transit peptide coding sequence that encodes a peptide that is useful for sub-cellular targeting of an operably linked protein, particularly to a chloroplast, leucoplast, or other plastid organelle; mitochondria; peroxisome; vacuole; or an extracellular location. Many chloroplast-localized proteins are expressed from nuclear genes as precursors and are targeted to the chloroplast by a chloroplast transit peptide (CTP). Examples of such isolated chloroplast proteins include, but are not limited to, those associated with the small subunit (SSU) of ribulose-1,5,-bisphosphate carboxylase, ferredoxin, ferredoxin oxidoreductase, the light-harvesting complex protein I and protein II, thioredoxin F, and enolpyruvyl shikimate phosphate synthase (EPSPS). Chloroplast transit peptides are described, for example, in U.S. Pat. No. 7,193,133. It has been demonstrated that non-chloroplast proteins may be targeted to the chloroplast by the expression of a heterologous CTP operably linked to the transgene encoding a non-chloroplast protein.

### Transcribable DNA Polynucleotides

As used herein, the term “transcribable DNA polynucleotide” refers to any DNA polynucleotide capable of being transcribed into an RNA, including, but not limited to, those having protein coding sequences (e.g., mRNAs), those encoding guide RNAs (gRNAs), and those producing RNAs having sequences useful for gene suppression (e.g., siRNAs, miRNAs, dsRNAs). The type of DNA polynucleotide can include, but is not limited to, a DNA polynucleotide from the same plant, a DNA polynucleotide from another plant, a DNA polynucleotide from a different organism, or a synthetic DNA polynucleotide, such as a DNA polynucleotide containing an antisense message of a gene, or a DNA polynucleotide encoding an artificial, synthetic, or otherwise modified version of a transgene. Examples of transcribable DNA polynucleotides for incorporation into constructs as described herein include, e.g., DNA polynucleotides or genes from a species other than the species into which the DNA polynucleotide is incorporated or genes that originate from, or are present in, the same species, but are incorporated into recipient cells by genetic engineering methods rather than classical breeding techniques.

As used herein, the term “heterologous transcribable DNA polynucleotide,” refers to a transcribable DNA polynucleotide that is heterologous with respect to one or more of the regulatory elements to which it is operably linked.

A “transgene” refers to a transcribable DNA polynucleotide heterologous to a host cell at least with respect to its location in the host cell genome and/or a transcribable DNA polynucleotide artificially incorporated into a host cell's genome in the current or any prior generation of the cell.

A regulatory element, such as a 3′ UTR (e.g., SEQ ID NO:1) or fragments or variants thereof, may be operably linked to a transcribable DNA polynucleotide that is heterologous with respect to the regulatory element. As used herein, the term “heterologous” refers to the combination of two or more DNA polynucleotides (or nucleotide sequences, or DNA sequences) when such a combination is not normally found in nature. For example, the two DNA polynucleotides (or nucleotide sequences, or DNA sequences) may be derived from different species and/or the two DNA polynucleotides (or nucleotide sequences, or DNA sequences) may be derived from different genes, e.g., different genes from the same species or the same genes from different species. A regulatory element is thus heterologous with respect to an operably linked transcribable DNA polynucleotide if such a combination is not normally found in nature, i.e., the transcribable DNA polynucleotide does not naturally occur operably linked to the regulatory element.

The transcribable DNA polynucleotide may generally be any DNA polynucleotide for which expression of a transcript is desired. Such expression of a transcript may result in translation of the resulting mRNA, and thus protein expression. Alternatively, for example, a transcribable DNA polynucleotide may be designed to ultimately cause decreased expression of a specific gene or protein. In one embodiment, this may be accomplished by using a transcribable DNA polynucleotide that is oriented in the antisense direction. One of ordinary skill in the art is familiar with using such antisense technology. Any gene may be negatively regulated in this manner, and, in one embodiment, a transcribable DNA polynucleotide may be designed for suppression of a specific gene through expression of a dsRNA, siRNA or miRNA.

Thus, one embodiment provides a recombinant DNA polynucleotide comprising a regulatory element, such as that provided as SEQ ID NO:1 or fragments or variants thereof, operably linked to a heterologous transcribable DNA polynucleotide so as to modulate transcription of the transcribable DNA polynucleotide at a desired level or in a desired pattern when the construct is integrated in the genome of a plant cell or a transgenic plant cell. In one embodiment, the transcribable DNA polynucleotide comprises a protein-coding region of a gene and in another embodiment the transcribable DNA polynucleotide comprises an antisense region of a gene.

### Genes of Agronomic Interest

A transcribable DNA polynucleotide may comprise a gene of agronomic interest. As used herein, the term “gene of agronomic interest” refers to a transcribable DNA polynucleotide that, when expressed in a particular plant tissue, cell, or cell type, confers a desirable characteristic. The product of a gene of agronomic interest may act within the plant in order to cause an effect upon the plant morphology, physiology, growth, development, yield, grain composition, nutritional profile, discase or pest resistance, and/or environmental or chemical tolerance or may act as a pesticidal agent in the dict of a pest that feeds on the plant. In one embodiment, a regulatory element such as that provided as SEQ ID NO:1 or fragments or variants thereof is incorporated into a construct such that the regulatory element is operably linked to a transcribable DNA polynucleotide that is a gene of agronomic interest. In a transgenic plant containing such a construct, the expression of the gene of agronomic interest can confer a beneficial agronomic trait. A beneficial agronomic trait may include, for example, but is not limited to, herbicide tolerance, insect control, modified yield, disease resistance, pathogen resistance, modified plant growth and development, modified starch content, modified oil content, modified fatty acid content, modified protein content, modified fruit ripening, enhanced animal and human nutrition, biopolymer productions, environmental stress resistance, pharmaceutical peptides, improved processing qualities, improved flavor, hybrid seed production utility, improved fiber production, augmented carbon sequestration, and/or desirable biofuel production.

Examples of genes of agronomic interest known in the art include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. USRE38,446; U.S. Pat. Nos. 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial discase resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,5412,59; 5,985,605; and 6,171,640), biopolymers (U.S. Pat. Nos. USRE37,543; U.S. Pat. Nos. 6,228,623; and 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

Alternatively, a gene of agronomic interest can affect the above mentioned plant characteristics or phenotypes by encoding an RNA that causes the targeted modulation of gene expression of an endogenous gene, for example by antisense RNA (see, e.g. U.S. Pat. No. 5,107,065); inhibitory RNA (“RNAi”) including modulation of gene expression by miRNA-, siRNA-, trans-acting siRNA-, and phased sRNA-mediated mechanisms, e.g., as described in published applications U.S. 2006/0200878 and U.S. 2008/0066206, and in U.S. patent application Ser. No. 11/974,469); or co-suppression-mediated mechanisms. The RNA could also be a catalytic RNA (e.g., a ribozyme or a riboswitch; see, e.g., U.S. 2006/0200878) engineered to cleave a desired endogenous mRNA product. Methods are known in the art for constructing and introducing constructs into a cell in such a manner that the transcribable DNA polynucleotide is transcribed into a molecule that is capable of causing gene suppression.

### Selectable Markers

Transcribable DNA polynucleotides encoding selectable markers may also be used with the regulatory element such as that provided as SEQ ID NO:1 or fragments or variants thereof. As used herein the term “selectable marker” refers to any transcribable DNA polynucleotide whose expression in a transgenic plant, tissue or cell, or lack thereof, can be screened for and/or scored in some way. Selectable markers (also referred to as reporter genes), and their associated selection and screening techniques, are known in the art and include, but are not limited to, transcribable DNA polynucleotides encoding β-glucuronidase (GUS), green fluorescent protein (GFP), proteins that confer antibiotic resistance, and proteins that confer herbicide tolerance. An example of a reporter transgene is provided as SEQ ID NO:3.

The use of reporter gene assays (also referred to as reporter transgene assays) to determine the gene regulatory activity (also referred to as the expression profile) of a regulatory element is well known in the art (e.g., Clark et al., Unit 4, Chapter 21 of Molecular Biology, Third Edition, Academic Press, Elsevier Inc., 2019). As used herein, the term “reporter gene assay” refers to a method in which first a reporter gene, such as a transgene encoding a β-glucuronidase (GUS) protein, is used as the heterologous transcribable DNA polynucleotide operably linked to a particular regulatory element to determine the gene regulatory activity (or the expression profile) of the latter e.g., a promoter or a 3′ UTR. In one embodiment, a reporter gene, such as a GUS gene, may be used in a reporter gene assay as the heterologous transcribable DNA polynucleotide operably linked to SEQ ID NO:1 or fragments or variants thereof, to determine the gene regulatory activity of SEQ ID NO:1. In the subsequent reporter gene assay, qualitative and quantitative GUS analysis may be used to evaluate the gene regulatory activity (or the expression profile) of a regulatory element in selected plant organs and/or tissues in transformed plants. It is understood that the gene regulatory activity (or the expression profile) of a regulatory element, e.g., a promoter or a 3′ UTR determined by using a reporter gene assay e.g., a GUS assay, is the same or substantially the same or substantially similar for other operably linked transcribable DNA polynucleotides besides GUS. In one embodiment, other operably linked transcribable DNA polynucleotides may be genes of agronomic interest, including, but not limited to, those described herein.

### Genome Editing

Several embodiments relate to a recombinant DNA construct comprising one or more expression cassette(s) comprising a sequence with at least about 85 percent sequence identity at least about 86 percent, at least about 87 percent, at least about 88 percent, at least about 89 percent, at least about 90 percent, at least 91 percent, at least 92 percent, at least 93 percent, at least 94 percent, at least 95 percent, at least 96 percent, at least 97 percent, at least 98 percent, or at least 99 percent sequence identity or more to SEQ ID NO:1 or a fragment or variant thereof, operably linked to a heterologous DNA sequence encoding a site-specific genome modification enzyme and/or any associated protein(s) to carry out genome modification. These site-specific genome modification enzyme-expressing cassette(s) may be present in the same molecule or vector as a donor template for templated editing (in cis) or on a separate molecule or vector (in trans). Several methods for editing are known in the art involving different sequence-specific genome modification enzymes (or complexes of proteins and/or guide RNA) that modify the genomic DNA. In some embodiments, a site-specific genome modification enzyme modifies the genome by inducing a double-strand break (DSB) or nick at a desired genomic site or locus. In some embodiments, during the process of repairing the DSB or nick introduced by the genome modification enzyme, a donor template DNA may become integrated into the genome at the site of the DSB or nick. In some embodiments, during the process of repairing the DSB or nick introduced by the genome modification enzyme, an insertion or deletion mutation (indel) may be introduced into the genome. In some embodiments, a site-specific genome modification enzyme comprises a cytidine deaminase. In some embodiments, a site-specific genome modification enzyme comprises an adenine deaminase. In the present disclosure, site-specific genome modification enzymes include endonucleases, recombinases, transposases, deaminases, helicases, reverse transcriptases and any combination thereof.

Several embodiments relate to a gene regulatory element as described herein operably linked to a heterologous transcribable DNA polynucleotide encoding one or more components of a genome editing system. Genome editing systems may be used to introduce one or more insertions, deletions, substitutions, base modifications, translocations, or inversions to a genome of a host cell. In some embodiments, a gene regulatory element as described herein is operably linked to a heterologous transcribable DNA polynucleotide encoding a sequence-specific DNA binding domain, such as a CRISPR-Cas effector protein, a zinc finger protein, or a transcription activator (TAL) protein. In some embodiments, the sequence-specific DNA binding domain maybe a fusion protein. In some embodiments, a gene regulatory element as described herein is operably linked to a heterologous transcribable DNA polynucleotide encoding a CRISPR-Cas effector protein. In some embodiments, the CRISPR-Cas effector protein is selected from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system. In some embodiments, a gene regulatory element as described herein is operably linked to a heterologous transcribable DNA polynucleotide encoding a guide RNA. As used herein, a “guide RNA” or “gRNA” refers to an RNA that recognizes a target DNA sequence and directs, or “guides”, a CRISPR effector protein to the target DNA sequence. A guide RNA is comprised of a region that is complementary to the target DNA (referred to as the CRISPR RNA or crRNA or the spacer) and a region that binds the CRISPR effector protein (referred to as the tracrRNA). A guide RNA may be a single RNA molecule (single guide RNA, “sgRNA”) or two separate RNA molecules (a dual-guide RNA, “dgRNA”). In some embodiments a gRNA may further comprise a prime editing (PE) guide RNA (“PEgRNA”) for a reverse transcriptase.

Several embodiments relate to a gene regulatory element as described herein operably linked to a heterologous transcribable DNA polynucleotide encoding one or more components of a CRISPR-Cas genome editing system comprising a CRISPR-Cas effector protein. Examples of CRISPR-Cas effector proteins include, but are not limited to, Cas9, Cas12b, C2c3, C2c4, C2c5, C2c8, C2c9, C2c10, Cas12a (also referred to as Cpf1), Cas12b, Cas12c, Cas12d, Cas12e, Cas12h, Cas12i, Cas12g, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Csc2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx 10, Csx 16, CsaX, Csx3, Csx1, Csx15, Csf1, Csf2, Csf3, Csf4 (dinG), Csf5, Cas14a, Cas 14b, and Cas 14c effector protein. In one embodiment, the CRISPR-Cas nuclease may be a Cas12a effector protein. In some embodiments, a gene regulatory element as described herein is operably linked to a CRISPR-Cas effector protein comprising a mutation in its nuclease active site (e.g., RuvC, HNH, and/or NUC domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation. In some embodiments, a gene regulatory element as described herein is operably linked to a CRISPR-Cas effector protein having a mutation in its nuclease active site to generate a nickase activity operably linked to a reverse transcriptase enzyme.

### Cell Transformation

Methods of producing transformed cells, plant cells and plants that comprise one or more regulatory elements, such as that provided as SEQ ID NO:1 or fragments or variants thereof, operably linked to a transcribable DNA polynucleotide are also provided.

The term “transformation” refers to the introduction of a DNA polynucleotide into a recipient host. As used herein, the term “host” refers to bacteria, fungi, or plants, including any cells, tissues, organs, or progeny of the bacteria, fungi, or plants. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign DNA polynucleotide, such as for example, a construct as described herein, has been introduced. The introduced DNA polynucleotide may be integrated into the genomic DNA of the recipient cell, tissue, organ, or organism such that the introduced DNA polynucleotide is inherited by subsequent progeny. A “transgenic” or “transformed” cell or organism may also include progeny of the cell or organism and progeny produced from a breeding program employing such a transgenic organism as a parent in a cross and exhibiting an altered phenotype resulting from the presence of a foreign DNA polynucleotide. The introduced DNA polynucleotide may also be transiently introduced into the recipient cell such that the introduced DNA polynucleotide is not inherited by subsequent progeny. The term “transgenic” refers to a bacterium, fungus, or plant containing one or more heterologous DNA polynucleotides.

There are many methods well known to those of skill in the art for introducing DNA polynucleotides into plant cells. The process generally comprises the steps of selecting a suitable host cell, transforming the host cell with a vector, and obtaining the transformed host cell. Methods and materials for transforming plant cells by introducing a plant construct into a plant genome can include any of the well-known and demonstrated methods. Suitable methods include, but are not limited to, bacterial infection (e.g., Agrobacterium), binary BAC vectors, direct delivery of DNA (e.g., by PEG-mediated transformation, desiccation/inhibition-mediated DNA uptake, electroporation, agitation with silicon carbide fibers, and acceleration of DNA coated particles), gene editing (e.g., CRISPR-Cas systems), among others.

Host cells may be any cell or organism, such as a plant cell, algal cell, algae, fungal cell, fungi, bacterial cell, or insect cell. In specific embodiments, the host cells and transformed cells may include cells from crop plants. In further specific embodiments, the host cells and transformed cells may include cells from soybean plants.

A transgenic plant subsequently may be regenerated from a transgenic plant cell as described herein. Using conventional breeding techniques or self-pollination, seed may be produced from this transgenic plant. Such seed, and the resulting progeny plant grown from such seed, will contain the recombinant DNA polynucleotide as described herein, such as that comprising the sequence presented as SEQ ID NO:1 or fragments or variants thereof, and therefore will be transgenic.

Transgenic plants can be self-pollinated to provide seed for homozygous transgenic plants (homozygous for a recombinant DNA polynucleotide as described herein) or crossed with non-transgenic plants or different transgenic plants to provide seed for heterozygous transgenic plants (heterozygous for a recombinant DNA polynucleotide as described herein). Both such homozygous and heterozygous transgenic plants are referred to herein as “progeny plants.” Progeny plants are transgenic plants descended from the original transgenic plant and containing a recombinant DNA polynucleotide as described herein. Seeds produced using a transgenic plant can be harvested and used to grow generations of transgenic plants, i.e., progeny plants comprising a recombinant DNA polynucleotide as described herein and expressing a gene of agronomic interest. Descriptions of breeding methods that are commonly used for different crops can be found in one of several reference books, see, e.g., Allard, Principles of Plant Breeding, John Wiley & Sons, NY, U. of CA, Davis, CA, 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

The transformed plants may be analyzed for the presence of the gene or genes of interest and the expression level and/or profile conferred by the regulatory element such as that provided as SEQ ID NO:1 or fragments or variants thereof. Those of skill in the art are aware of the numerous methods available for the analysis of transformed plants. For example, methods for plant analysis include, but are not limited to, Southern blots or northern blots, PCR-based approaches, biochemical analyses, phenotypic screening methods, field evaluations, and immunodiagnostic assays. The expression of a transcribable DNA polynucleotide can be measured using TaqMan® (Applied Biosystems, Foster City, CA) reagents and methods as described by the manufacturer and PCR cycle times determined using the TaqMan® Testing Matrix. Alternatively, the Invader® (Third Wave Technologies, Madison, WI) reagents and methods as described by the manufacturer can be used to evaluate transgene expression.

Also provided are parts of a plant as described herein. Plant parts include, but are not limited to, leaves, stems, roots, tubers, seeds, endosperm, ovule, and pollen. Plant parts may be viable, nonviable, regenerable, and/or non-regenerable. Also provided are transformed plant cells comprising a DNA polynucleotide as described herein, such as that provided as SEQ ID NO:1 or fragments or variants thereof. The transformed or transgenic plant cells include regenerable and/or non-regenerable plant cells.

Commodity products that are produced from a transgenic plant or part thereof may contain the recombinant DNA polynucleotide as described herein, such as that provided as SEQ ID NO:1 or fragments or variants thereof. In some embodiments, commodity products may contain a detectable amount of DNA comprising the DNA sequence presented as SEQ ID NO:1 or fragments or variants thereof. As used herein, a “commodity product” refers to any composition or product which is comprised of material derived from a transgenic plant, seed, plant cell, or plant part containing the recombinant DNA polynucleotide as described herein, such as that provided as SEQ ID NO:1 or fragments or variants thereof. Commodity products include but are not limited to processed seeds, grains, plant parts, and meal. A commodity product containing a detectable amount of DNA corresponding to the recombinant DNA polynucleotide as described herein, such as that provided as SEQ ID NO:1 or fragments or variants thereof is contemplated. Detection of one or more of this DNA in a sample may be used for determining the content or the source of the commodity product. Any standard method of detection for DNA polynucleotides may be used, including methods of detection disclosed herein.

The definitions and methods provided define the present disclosure and guide those of ordinary skill in the art in the practice of the present disclosure. Unless otherwise noted, terms are to be understood according to conventional usage by those of ordinary skill in the relevant art. Definitions of common terms and methods in molecular biology may also be found, for example, in Clark et al., Molecular Biology, Third Edition, Academic Press, Elsevier Inc., 2019; Alberts et al., Molecular Biology of The Cell, 5th Edition, Garland Science Publishing, Inc.: New York, 2007; Rieger et al., Glossary of Genetics: Classical and Molecular, 5th edition, Springer-Verlag: New York, 1991; King et al., A Dictionary of Genetics, 6th ed., Oxford University Press: New York, 15 2247; and Lewin, Genes IX, Oxford University Press: New York, 2007.

### EMBODIMENTS

For further illustration, additional exemplary, non-limiting embodiments of the present disclosure are set forth below.

Embodiment 1 is a recombinant DNA polynucleotide comprising a DNA sequence selected from the group consisting of:
a) a sequence with at least 85 percent sequence identity to SEQ ID NO:1;
b) a sequence comprising SEQ ID NO:1; and
c) a fragment of SEQ ID NO:1, wherein the fragment comprises gene regulatory activity;
   wherein said DNA sequence is operably linked to a heterologous transcribable DNA polynucleotide.

Embodiment 2 is the recombinant DNA polynucleotide of embodiment 1, wherein the DNA sequence has at least 90 percent sequence identity to the DNA sequence of SEQ ID NO:1.

Embodiment 3 is the recombinant DNA polynucleotide of any one of embodiments 1 or 2, wherein the DNA sequence has at least 95 percent sequence identity to the DNA sequence of SEQ ID NO: 1.

Embodiment 4 is the recombinant DNA polynucleotide of any one of embodiments 1 to 3, wherein the DNA sequence comprises gene regulatory activity.

Embodiment 5 is the recombinant DNA polynucleotide of any one of embodiments 1 to 4, wherein the heterologous transcribable DNA polynucleotide comprises a gene of agronomic interest.

Embodiment 6 is the recombinant DNA polynucleotide of embodiment 5, wherein the gene of agronomic interest confers herbicide tolerance in plants.

Embodiment 7 is the recombinant DNA polynucleotide of embodiment 5, wherein the gene of agronomic interest confers pest resistance in plants.

Embodiment 8 is the recombinant DNA polynucleotide of any one of embodiments 1 to 4, wherein the heterologous transcribable DNA polynucleotide encodes a dsRNA, a miRNA, or a siRNA.

Embodiment 9 is a transgenic plant cell comprising a recombinant DNA polynucleotide comprising a DNA sequence selected from the group consisting of:
a) a sequence with at least 85 percent sequence identity to SEQ ID NO:1;
b) a sequence comprising SEQ ID NO:1; and
c) a fragment of SEQ ID NO:1, wherein the fragment comprises gene regulatory activity;
   wherein said DNA sequence is operably linked to a heterologous transcribable DNA polynucleotide.

Embodiment 10 is the transgenic plant cell of embodiment 9, wherein the transgenic plant cell is a monocotyledonous plant cell.

Embodiment 11 is the transgenic plant cell of embodiment 9, wherein the transgenic plant cell is a dicotyledonous plant cell.

Embodiment 12 is a transgenic plant, or part thereof, comprising the recombinant DNA polynucleotide of any one of embodiments 1 to 8.

Embodiment 13 is a progeny plant of the transgenic plant of embodiment 12, or a part thereof, wherein the progeny plant or part thereof comprises the recombinant DNA polynucleotide of any one of embodiments 1 to 8.

Embodiment 14 is a transgenic seed, wherein the seed comprises the recombinant DNA polynucleotide of any one of embodiments 1 to 8.

Embodiment 15 is a method of producing a commodity product comprising obtaining a transgenic plant or part thereof according to embodiment 12 or a progeny plant or part thereof according to embodiment 13 and producing the commodity product therefrom.

Embodiment 16 is the method of embodiment 15, wherein the commodity product is selected from the group consisting of seeds, processed seeds, protein concentrate, protein isolate, starch, grains, plant parts, seed oil, biomass, flour, and meal.

Embodiment 17 is a method of method of producing a transgenic plant according to embodiment 12 comprising
a) transforming a plant cell with the recombinant DNA polynucleotide of any one of embodiments 1 to 8 to produce a transformed plant cell; and
b) regenerating a transgenic plant from the transformed plant cell.

Embodiment 18 is a method of expressing a transcribable DNA polynucleotide comprising obtaining a transgenic plant according to embodiment 12 or a progeny plant or part thereof according to embodiment 13 and cultivating said plant, wherein the transcribable DNA polynucleotide is expressed.

Embodiment 19 is an isolated recombinant DNA molecule, characterized by comprising a DNA sequence of SEQ ID NO:1, wherein said DNA sequence is operably linked to a heterologous transcribable polynucleotide molecule.

Embodiment 20 is the isolated recombinant DNA molecule of embodiment 19, characterized in that the heterologous transcribable polynucleotide molecule comprises a gene of agronomic interest.

Embodiment 21 is the isolated recombinant DNA molecule of embodiment 20, characterized in that the gene of agronomic interest confers herbicide tolerance in plants.

Embodiment 22 is the isolated recombinant DNA molecule of embodiment 20, characterized in that the gene of agronomic interest confers pest resistance in plants.

Embodiment 23 is a method of producing a transgenic plant, excluding the plant obtained by said method, characterized by comprising:
a) transforming a plant cell with the isolated recombinant DNA molecule of embodiment 19 to produce a transformed plant cell; and
b) regenerating a transgenic plant from the transformed plant cell.

Embodiment 24 is a construct characterized by comprising the isolated recombinant DNA molecule of embodiment 19.

The embodiments described herein may be more readily understood through reference to the following examples, which are provided by way of illustration, and are not intended to be limiting, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1

### Design, Synthesis, and Cloning of the Synthetic 3′ UTR, T-Zm.GST314 (SEQ ID NO:1)

A novel, synthetic transcriptional regulatory element such as the synthetic 3′ UTR, T-Zm.GST314 (SEQ ID NO:1), is a synthetic expression element designed through algorithmic methods. This computationally derived synthetic transcriptional regulatory element was chemically synthesized and cloned using skills known in the art. Well over several hundreds of synthetic 3′ UTRs were designed and assayed in protoplasts and stably transformed plants to identify those synthetic 3′ UTRs that provided desired characteristics such as protein expression levels, patterns of expression, and proper termination and polyadenylation of the transcript. The synthetic 3′ UTR of the present disclosure was characterized for its effect on gene expression as well as proper termination of the transcript of the operably linked heterologous transcribable DNA polynucleotide.

The designed synthetic 3′ UTR, T-Zm.GST314 (SEQ ID NO:1), does not have extended homology to any known nucleic acid sequences that exist yet affects transcription of an operably linked coding sequence in the same manner as naturally occurring 3′ UTRs. The synthetic 3′ UTR, T-Zm.GST314, was cloned using methods known in the art into binary plant transformation vectors, operably linked to a β-glucuronidase (GUS) coding sequence, and the levels and patterns of expression in stably transformed corn plants were evaluated.

### Example 2

### Analysis of the Synthetic 3′ UTR, T-Zm.GST314 (SEQ ID NO:1): Its Effect on Expression of a Transgene in Stably Transformed Soybean Plants

Soybean plants were transformed with a plant binary expression vector construct containing transcriptional regulatory elements driving expression of the β-glucuronidase (GUS) transgene and the synthetic 3′ UTR, T-Zm.GST314 (SEQ ID NO:1) presented in Example 1. The resulting plants were analyzed for GUS protein expression, to assess the effect of the synthetic 3′ UTR, T-Zm.GST314, on transgene expression.

Soybean plants were transformed with plant binary GUS expression vector constructs. The synthetic 3′ UTR, T-Zm.GST314, was cloned into a base plant expression vector using methods known in the art. The resulting plant expression vector contained a left border region from Agrobacterium tumefaciens, a first transgene selection cassette used for selection of transformed plant cells that confers resistance to the antibiotic spectinomycin; a second transgene cassette to assess the activity of the synthetic 3′ UTR, T-Zm.GST314, which comprised the EXP, EXP-At.Cyco (SEQ ID NO:2), operably linked 5′ to a synthetic coding sequence designed for expression in a plant cell encoding β-glucuronidase (GUS, SEQ ID NO:3) containing a processable intron derived from the potato light-inducible tissue-specific ST-LSI gene (GenBank Accession: X04753), operably linked 5′ to the synthetic 3′ UTR T-Zm.GST314 (SEQ ID NO:1), followed by a right border region from Agrobacterium tumefaciens. A GUS construct used as a control was also used to transform soybean plants, which comprised an expression cassette using the same EXP-At.Cyco (SEQ ID NO:2) driving GUS expression wherein instead, the 3′ UTR, T-Gb.FbL2 (SEQ ID NO:4) was operably linked 3′ to the GUS synthetic coding sequence (SEQ ID NO:3).

Soybean plant cells were transformed using the binary transformation vector construct described above by Agrobacterium-mediated transformation, as is well known in the art. The resulting transformed plant cells were induced to form whole soybean plants.

Qualitative and quantitative GUS analysis was used to evaluate expression element activity in selected plant organs and tissues in transformed plants. For qualitative analysis of GUS expression by histochemical staining, whole-mount or sectioned tissues were incubated with GUS staining solution containing 1 mg/mL of X-Gluc (5-bromo-4-chloro-3-indolyl-b-glucuronide) for 5 h at 37° C. and de-stained with 35% EtOH and 50% acetic acid. Expression of GUS was qualitatively determined by visual inspection of selected plant organs or tissues for blue coloration under a dissecting or compound microscope.

For quantitative analysis of GUS expression by enzymatic assays, total protein was extracted from selected tissues of transformed soybean plants. One to two micrograms of total protein were incubated with the fluorogenic substrate, 4-methylumbelliferyl-β-D-glucuronide (MUG) at 1 mM concentration in a total reaction volume of 50 microliters. After 1 h incubation at 37° C., the reaction was stopped by adding 350 microliters of 200 mM sodium bicarbonate solution. The reaction product, 4-methylumbelliferone (4-MU), is maximally fluorescent at high pH, where the hydroxyl group is ionized. Addition of the basic sodium carbonate solution simultaneously stops the assay and adjusts the pH for quantifying the fluorescent product 4-MU. The amount of 4-MU formed was estimated by measuring its fluorescence using a FLUOstar Omega Microplate Reader (BMG LABTECH) (excitation at 355 nm, emission at 460 nm). GUS activity values (i.e., GUS expression) are provided in nmoles of 4-MU/hour/mg total protein.

The following tissues were sampled for GUS expression in the Ro generation: V5 stage Sink Leaf, Source Leaf, and Root; R1 stage Flowers, Petioles, Source Leaf, Pollen, and Root; R3 stage Pod and Immature Seed; R5 Source Leaf; and R8 Seed Cotyledon and Embryo. Soybean vegetative and reproductive stages are well known to those of skill in the art and numerous publications describing these stages can be found on the world wide web and elsewhere, such as North Dakota State University Publication A-1174 June 1999, Reviewed and Reprinted August 2004. Table 1 shows the range and mean GUS expression for each of the constructs.

TABLE 1
Effect of 3′ UTR regulatory elements on the quantitative
GUS expression in stably transformed R₀ soybean plants.

| | | EXP-At.Cyco/T- | | EXP-At.Cyco/T- | |
|---|---|---|---|---|---|
| | | Zm.GST314 | | Gb.FbL2:3 | |
| | | (SEQ ID NO: 1) | | (SEQ ID NO: 4) | |
| Stage | Organ | Range | Mean | Range | Mean |
| V5 | Sink Leaf | 21.21-44.95 | 28.28 | 31.31-77.85 | 48.3 |
| | Source Leaf | 20.25-27.72 | 23.56 | 21.54-42.73 | 28.75 |
| | Root | 21.31-65.04 | 47.51 | 51.72-121.62 | 95.53 |
| R1 | Flowers | 46.56-64.3 | 54.26 | 42.82-197.52 | 112.52 |
| | Petiole | 29.99-136.24 | 69.41 | 153.59-241.32 | 204.93 |
| | Source Leaf | 21.63-32.26 | 26.94 | 28.82-47.06 | 38.97 |
| | Pollen | 26.49-75.59 | 59.88 | 287.9-377.41 | 328.22 |
| | Root | 22.21-65.83 | 48.69 | 73.8-205.14 | 128.16 |
| R3 | Pod | 30.87-854.33 | 177.09 | 132.85-273.94 | 191.81 |
| | Immature | 31.74-100.33 | 57.64 | 69.83-411.77 | 164.88 |
| | Seed | | | | |
| R5 | Source Leaf | 20.12-69.61 | 35.94 | 29.61-42.67 | 37.21 |
| R8 | Seed | 20.79-397.74 | 137.41 | 20.21-763.51 | 234.44 |
| | Cotyledon | | | | |
| | Seed | 23.14-375.2 | 166.15 | 20.46-645.45 | 225.79 |
| | Embryo | | | | |

As can be seen in Table 1 above, the synthetic 3′ UTR, T-Zm.GST314 (SEQ ID NO:1), affected expression of the GUS transgene in a manner different than that of the 3′ UTR, T-Gb.FbL2 (SEQ ID NO:4). Expression was lower in most tissues in events transformed with the construct that comprised T-Zm.GST314 (SEQ ID NO:1) when compared to events that comprised T-Gb.FbL2 (SEQ ID NO:4), except for V5, R1 and R5 Source Leaf and R3 Pod, where expression was similar with both 3′ UTRs. Lower expression may be desired when expressing transgenes that may cause deleterious effects if expressed too high in the transgenic plant. Expression in R1 Pollen was more than five-fold less using T-Zm.GST314 (SEQ ID NO:1) when compared to the control T-Gb.FbL2 (SEQ ID NO:4). Lower pollen expression is particularly important when expressing such transgenes as pesticidal insect toxin genes that might be active against non-target insect pests that feed on pollen. Analysis of the GUS transcripts comprising T-Zm.GST314 (SEQ ID NO:1) demonstrated proper and efficient termination of transcription and polyadenylation (data not shown). Thus, the 3′ UTR, T-Zm.GST314 (SEQ ID NO:1) performs in a comparable manner as a naturally occurring 3′ UTR that is, it is able to affect expression of a transgene, and it is able to properly terminate the transcription of a transgene operably linked to it.

Having illustrated and described the principles of the present invention, it should be apparent to persons skilled in the art that the invention can be modified in arrangement and detail without departing from such principles. All publications and published patent documents cited herein are hereby incorporated by reference to the same extent as if cach individual publication or patent application is specifically and individually indicated to be incorporated by reference.

What is claimed is:

1. A recombinant DNA polynucleotide comprising a DNA sequence selected from the group consisting of:
a) a sequence with at least 85 percent sequence identity to SEQ ID NO:1;
b) a sequence comprising SEQ ID NO:1; and
c) a fragment of SEQ ID NO:1, wherein the fragment comprises gene regulatory activity;
wherein said DNA sequence is operably linked to a heterologous transcribable DNA polynucleotide.

2. The recombinant DNA polynucleotide of claim 1, wherein the DNA sequence has at least 90 percent sequence identity to the DNA sequence of SEQ ID NO:1.

3. The recombinant DNA polynucleotide of claim 1, wherein the DNA sequence has at least 95 percent sequence identity to the DNA sequence of SEQ ID NO:1.

4. The recombinant DNA polynucleotide of claim 1, wherein the DNA sequence comprises gene regulatory activity.

5. The recombinant DNA polynucleotide of claim 1, wherein the heterologous transcribable DNA polynucleotide comprises a gene of agronomic interest.

6. The recombinant DNA polynucleotide of claim 5, wherein the gene of agronomic interest confers herbicide tolerance in plants.

7. The recombinant DNA polynucleotide of claim 5, wherein the gene of agronomic interest confers pest resistance in plants.

8. The recombinant DNA polynucleotide of claim 1, wherein the heterologous transcribable DNA polynucleotide encodes a dsRNA, a miRNA, or a siRNA.

9. A transgenic plant cell comprising a recombinant DNA polynucleotide comprising a DNA sequence selected from the group consisting of:
a) a sequence with at least 85 percent sequence identity to SEQ ID NO:1;
b) a sequence comprising SEQ ID NO:1; and
c) a fragment of SEQ ID NO:1, wherein the fragment comprises gene regulatory activity;
wherein said DNA sequence is operably linked to a heterologous transcribable DNA polynucleotide.

10. The transgenic plant cell of claim 9, wherein the transgenic plant cell is a monocotyledonous plant cell.

11. The transgenic plant cell of claim 9, wherein the transgenic plant cell is a dicotyledonous plant cell.

12. A transgenic plant, or part thereof, comprising the recombinant DNA polynucleotide of claim 1.

13. A progeny plant of the transgenic plant of claim 12, or a part thereof, wherein the progeny plant or part thereof comprises the recombinant DNA polynucleotide of claim 1.

14. A transgenic seed, wherein the seed comprises the recombinant DNA polynucleotide of claim 1.

15. A method of producing a commodity product comprising obtaining a transgenic plant or part thereof according to claim 12 and producing the commodity product therefrom.

16. The method of claim 15, wherein the commodity product is selected from the group consisting of seeds, processed seeds, protein concentrate, protein isolate, starch, grains, plant parts, seed oil, biomass, flour, and meal.

17. A method of producing a transgenic plant according to claim 12 comprising
a) transforming a plant cell with the recombinant DNA polynucleotide of claim 1 to produce a transformed plant cell and
b) regenerating a transgenic plant from the transformed plant cell.

18. A method of expressing a transcribable DNA polynucleotide comprising obtaining a transgenic plant according to claim 12 and cultivating said plant, wherein the transcribable DNA polynucleotide is expressed.
